(19) United States
(12) Patent Application Publication
(10) Pub. No.: US 2014/0322723 A1
(43) Pub. Date: Oct. 30, 2014

(54) DIABETES DIAGNOSIS THROUGH THE DETECTION OF GLYCATED PROTEINS IN URINE

(71) Applicant: President and Dellows of Harvard College, Cambridge, MA (US)

(72) Inventors: Jose A. Halperin, Brookline, MA (US); Michael Chorev, Chestnut Hill, MA (US)

(21) Appl. No.: 14/359,460

(22) PCT Filed: Nov. 19, 2012
(86) PCT No.: PCT/US12/65811

§ 371 Date: May 20, 2014

Related U.S. Application Data
(60) Provisional application No. 61/562,630, filed on Nov. 22, 2011.

(51) Int. Cl. G01N 33/68

(52) U.S. Cl. CPC G01N 33/6893
USPC 435/7.1; 436/501

(57) ABSTRACT

The invention involves methods, compositions, and kits for detecting glycated proteins in a sample (e.g., urine or other bodily fluid) from a subject. Also provided are methods, compositions, and kits for diagnosing or following a diabetic condition of the subject or screening for a diabetic condition in a population of subjects based upon the detection of the glycated protein(s) in the sample.

### RELATED APPLICATIONS

The present application claims priority under 35 U.S.C. §119(e) to U.S. provisional patent application, U.S. Ser. No. 61/562,630, filed Nov. 22, 2011, which is incorporated herein by reference.

### BACKGROUND OF THE INVENTION

Diabetes mellitus is a leading cause of morbidity and mortality in the adult population. This is primarily because diabetic patients tend to develop vascular complications that involve the kidneys (diabetic nephropathy), the retina (diabetic retinopathy), as well as large and small blood vessels in other organs (macro- and microvascular disease) including nerves (diabetic neuropathy). It is well established that the vascular complications of diabetes are caused by elevated blood glucose levels over long periods of time. Elevated blood glucose levels affect proteins by a process known as glycation.

Glycation, the non-enzymatic attachment of glucose to proteins, is considered a major pathophysiological mechanism causing tissue damage in diabetic subjects. Glycation involves the reaction of glucose and/or other reducing sugars with amino groups in proteins resulting in the formation of a Schiff base or aldimine. This labile adduct can tautomerize via the Amadori rearrangement to form the more stable ketoamine. The ketoamine may further undergo cyclization or oxidation to yield β-hydroxylamine or β,γ-diketoamine, respectively. The ketoamine and β-hydroxylamine are respectively the linear and cyclic forms of the Amadori product. Shown below in the reaction scheme is an exemplary glycation process.

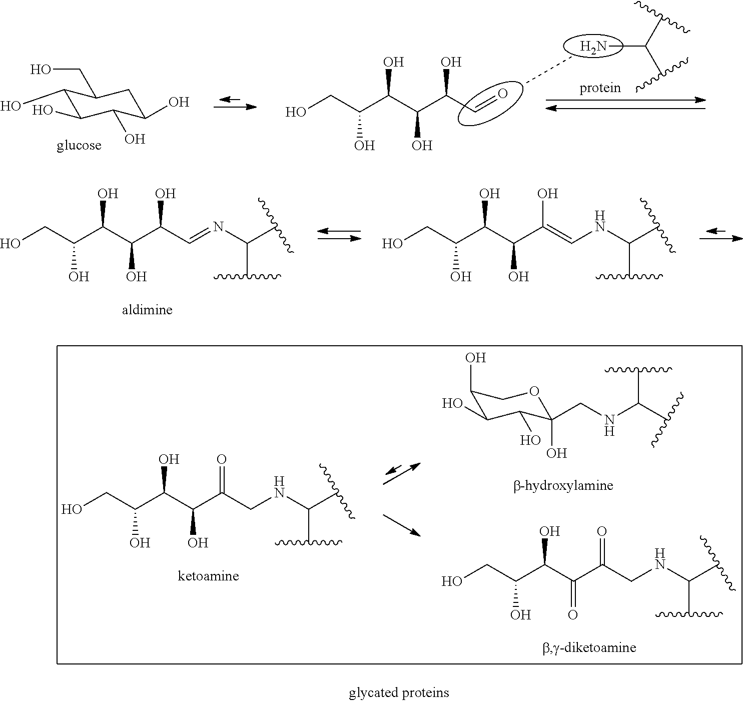

The function of the glycated protein may be impaired, depending on the location of the amino group(s) affected. For example, N-terminal glycation of the β-chains of hemoglobin gives rise to glycated hemoglobin in which responsiveness to 2,3-diphosphoglycerate is decreased and oxygen affinity is increased. Glycation of the major thrombin inhibitor of the coagulation system, antithrombin III, decreases its affinity for heparin, and has been postulated to contribute to the hypercoagulable state associated with diabetes.

Currently, protein glycation in diabetic subjects is measured in blood by estimating the amount of glycated hemoglobin through a clinical test that requires a blood sample. Accordingly, there is a need for a simplified and less invasive method for monitoring of protein glycation levels.

### SUMMARY OF THE INVENTION

The present invention provides for novel methods, compositions, and kits for detecting glycated proteins in a sample from a subject. The sample may be urine, blood, sweat, plasma, serum, saliva, or other bodily fluids. In certain embodiments, the subject is a urine sample. The subject may be a mammal, including a human.

It has been found that the level of glycated proteins is elevated in diabetic patients and that glycated proteins are present in urine and other bodily fluids of diabetic patients. Therefore, by detecting glycated proteins in a sample, the methods, compositions, and kits of the present invention can be used to diagnose, follow the diabetic condition of a subject previously diagnosed with a diabetic condition, or to screen for diabetes in a population of subjects.

The present invention, in one aspect, provides for methods of diagnosing a diabetic condition in a subject. The method of diagnosing includes detecting one or more glycated proteins in a sample obtained from the subject. If the glycated protein(s) are detected in the sample above a threshold level, the subject is diagnosed with having a diabetic condition. If the glycated protein(s) are not detected in the sample or are detected below a threshold level, the subject is not diagnosed with having a diabetic condition. The threshold level may be a predetermined value. For example, the threshold level may also be the level of glycated protein(s) in a control sample obtained from a subject. The control sample may be a sample obtained from a normal subject, that is a subject who does not have diabetic condition.

In the present invention, the glycated protein(s) being detected in the sample may be glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, glycated apolipoprotein A4, or combinations thereof. Other glycated protein(s) that may be detected in the present invention include, but are not limited to, glycated CD59, glycated albumin, glycated hemoglobin, other glycated proteins found in urine, and combinations thereof.

The present invention can be used to detect any number of glycated proteins in a sample. Two, three, four, five, or even more glycated proteins may be assayed for in a sample from a subject.

Glycated proteins are commonly found in plasma. Some of those glycated proteins, especially ones of low molecular weight(s), may pass through the filtration membrane in the glomerulus and reach the urine. Glycated protein(s) in urine being detected in the present invention have a molecular weight of less than approximately 30,000 g/mol. The present invention can also be used to detect glycated protein(s) having other molecular weight(s) as long as the glycated protein(s) are found in the urine.

Glycated protein(s) may be detected using an antibody, or antigen-binding fragment thereof, that binds to one or more glycated proteins. Other techniques known in the art for analyzing proteins may also be used to detect glycated proteins, for example, MS, LC-MS, and HPLC. The binding may be specific or non-specific. The antibody may be a monoclonal or polyclonal antibody.

The antibody of the present invention may bind to a glycated residue of a protein. Residues likely to be glycated typically have one or more nucleophilic groups, such as amino groups. Exemplary residues include lysine, arginine, and the N-terminus.

Glycated protein(s) may be detected using an immunoassay, such as a sandwich-type assay, competitive binding assay, one-step direct test, two-step test, dot blot assay, or reverse dot blot assay.

The subject being tested may have been previously diagnosed with a diabetic condition, or may have never been diagnosed with a diabetic condition. The diabetic condition may be characterized by abnormal level(s) of glycated protein(s) in a bodily fluid, hyperglycemia, impaired glucose tolerance, insulin resistance, hepatic steatosis, non-alcoholic steatohepatitis (NASH), pediatric NASH, obesity, childhood obesity, metabolic syndrome, polycystic ovary disease, gestational diabetes, or combinations thereof.

The subject of the present invention may have been previously diagnosed or have not been previously diagnosed with a pre-diabetic condition. The pre-diabetic condition may be characterized by abnormal level(s) of glycated protein(s) in a bodily fluid, metabolic syndrome, impaired glucose tolerance, impaired fasting glycemia, and combinations thereof.

In the present invention, the subject may or may not be treated to regulate blood sugar levels.

In another aspect, provided are methods of diagnosing a diabetic condition in a subject by obtaining the level of one or more glycated proteins in a sample obtained from the subject. If the level of the glycated protein(s) exceeds a threshold level, the subject is diagnosed with having a diabetic condition. In certain embodiments, the level of glycated protein(s) is 1.2-fold, 1.5-fold, 2-fold, 3-fold, 4-fold, 5-fold, 10-fold, or more above the threshold level to be diagnosed with a diabetic condition. If the level of the glycated protein(s) is below the threshold level, the subject is not diagnosed with having a diabetic condition.

In yet another aspect, the present invention provides methods of diagnosing a diabetic condition in a subject. First, the level of one or more glycated proteins in a test sample from the subject is obtained. The level of the glycated protein(s) in the test sample is then compared to the level of glycated protein(s) in a control sample (e.g., a sample from a non-diabetic patient). In yet another aspect, provided are methods of following a subject with a diabetic condition by detecting one or more glycated proteins in a sample obtained from the subject. The levels of the glycated protein(s) in the sample indicate the onset, progression, or regression of the diabetic condition.

In yet another aspect, the present invention provides methods of screening a population of subjects for a diabetic condition. The methods of screening include detecting one or more glycated protein(s) in a sample obtained from each subject. If the glycated protein(s) are detected in a sample of a subject above a specified threshold level, the subject is diagnosed with having a diabetic condition. If the glycated protein(s) are not detected in a sample of a subject or are below a threshold level, the subject is not diagnosed with having a diabetic condition.

In yet another aspect, the present invention includes a composition of an antibody or collection of antibodies useful in detecting glycated protein(s) in a sample from a subject.

In still another aspect, provided for is a kit useful in performing the methods of the present invention. In certain embodiments, the kit includes an antibody or collection of antibodies, instructions for using the antibody or collection of antibodies to detect glycated protein(s), one or more containers, or combinations thereof.

### DEFINITIONS

A “subject” for which diagnosis, testing, administration, and/or other medical evaluation or treatment are contemplated includes, but is not limited to, humans (i.e., a male or female of any age group, e.g., a pediatric subject (e.g, infant, child, adolescent) or adult subject (e.g., young adult, middle-aged adult or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs), birds (e.g., commercially relevant birds such as chickens, ducks, geese, and/or turkeys), reptiles, amphibians, and fish. In certain embodiments, the non-human animal is a mammal. The non-human animal may be a male or female at any stage of development. A non-human animal may be a transgenic animal.

As used herein, “diabetes,” or “diabetes mellitus,” is a group of metabolic diseases in which a subject has high blood sugar levels, either because the body of the subject does not produce enough insulin, or because cells in the body do not respond to the insulin that is produced by the body. These high blood sugar levels produce the classical symptoms of polyuria (frequent urination), polydipsia (increased thirst), and polyphagia (increased hunger). There are three main types of diabetes. Type 1 diabetes results from the body's failure to produce insulin and presently requires the subject to inject insulin. Type 2 diabetes results from insulin resistance, a condition in which cells fail to use insulin properly, sometimes combined with an absolute insulin deficiency. Some women develop gestational diabetes, a third type of diabetes, in the middle to late stages of pregnancy. Gestational diabetes is caused by the hormones of pregnancy or a shortage of insulin.

The term “polypeptide” refers to a polymer of amino acids without regard to the length of the polymer; thus, “peptides,” “oligopeptides”, and “proteins” are included within the definition of polypeptide and used interchangeably herein. This term also does not specify or exclude chemical or post-translational modifications of the polypeptides. Therefore, for example, modifications to polypeptides that include the covalent attachment of glycosyl groups, acetyl groups, phosphate groups, lipid groups, and the like are expressly encompassed by the term polypeptide. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. See, for instance, Proteins-structure and molecular properties, 2^{nd} Ed., T. E. Creighton, W. H. Freeman and Company, New York, 1993; Posttranslational covalent modification of proteins, Johnson, Ed., Academic Press, New York, pp. 1-12, 1983; Seifter et al., Meth. Enzymol. (1990) 182:626-646; Rattan et al., Ann. NY Acad. Sci. (1992) 663:48-62. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

As used herein, “glycation” refers to the non-enzymatic modification of a protein or lipid molecule with a reducible sugar molecule, such as fructose or glucose. Glycation may occur inside or outside the body of a subject. Glycation is a process that may impair the functioning of biomolecules.

“Glucitollysine,” as used herein, refers to (2S)-2-amino-6-[[(2S,3R,4R,5R)-2,3,4,5,6-pentahydroxyhexyl]amino]hexanoic acid. See, e.g., Day et al., J. Biol. Chem. (1979) 254:9394-9400; Robins et al., Biochem. Biophys. Res. Commun. (1972) 48:76-84; Felix et al., J. Org. Chem. (1978) 43:4194-4196.

By “isolated,” it is meant separated from its native environment and present in sufficient quantity to permit its identification or use according to the procedures described herein. The term “isolated” includes (1) selectively produced by expression cloning; or (2) purified as by immunoprecipitation, chromatography, or electrophoresis. Because an isolated material may be admixed with a carrier in a preparation, such as, for example, for injecting into a subject, the isolated material may comprise only a small percentage by weight of the preparation. The material is nonetheless isolated in that it has been separated from the substances with which it typically is associated in living systems.

The term “purified,” as applied to proteins herein, refers to a composition wherein the desired protein comprises at least 35% of the total protein component in the composition. The desired protein preferably comprises at least 40%, more preferably at least about 50%, more preferably at least about 60%, still more preferably at least about 70%, even more preferably at least about 80%, even more preferably at least about 90%, and most preferably at least about 95% of the total protein component. The composition may contain other compounds such as carbohydrates, salts, lipids, solvents, and the like, without affecting the determination of the percentage purity as used herein.

As used herein, the term “immunoglobulin” refers to a protein consisting of one or more polypeptides substantially encoded by immunoglobulin genes. One form of immunoglobulin constitutes the basic structural unit of an antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions.

The term “antibody,” as used herein, refers to an intact antibody or a fragment of an antibody that competes with the intact antibody for antigen binding. Antibody fragments include, but are not limited to, Fab, Fab′, F(ab′)₂, Fv, scFv, Fd, diabodies, and other antibody fragments that retain at least a portion of the variable region of an intact antibody (see, e.g., Hudson et al. (2003) Nat. Med. 9:129-134). In certain embodiments, antibody fragments are produced by enzymatic or chemical cleavage of intact antibodies. In certain embodiments, antibody fragments are produced by recombinant DNA techniques.

As used herein, the term “antigen” refers to a substance or molecule that, when introduced as is or conjugated to carrier proteins such as KLH or synthetic carriers such as dendrimers with or without an adjuvant into the body of a subject, triggers the production of an antibody by the immune system, which will then kill or neutralize the antigen that is recognized by the antibody. The term “antigen” may also refer to any molecule or molecular fragment that can be bound by a major histocompatibility complex (MHC) and presented to a T-cell receptor. “Self” antigens are usually tolerated by the immune system; whereas “non-self” antigens are identified as foreign and attacked by the immune system.

As used herein, “binding specifically” means being capable of distinguishing a material from other materials sufficient for the purpose to which the present invention relates. Thus, “binding specifically to a glycated protein” means being capable of distinguishing the glycated protein from other molecules. In certain embodiments, the antibody, or antigen-binding fragment thereof, binds to one particular glycated protein and does not bind to any other glycated proteins. In certain embodiments, the antibody, or antigen-binding fragment thereof, binds to two or more glycated proteins, of which the binding affinity for one glycated protein bound is substantially higher than the binding affinity for any other glycated proteins bound. In certain embodiments, the antibody, or antigen-binding fragment thereof, binds to two or more glycated proteins, of which the binding affinity for one glycated protein bound is substantially lower than the binding affinity for any other glycated proteins bound.

As used herein, “binding non-specifically” means being incapable of distinguishing a material from other materials sufficient for the purposes of the present invention. Thus, “binding non-specifically to a glycated protein” means being incapable of distinguishing the glycated protein from other materials. For example, the antibody, or antigen-binding fragment thereof, may bind to two or more glycated proteins with similar binding affinities. The antibody may, however, be able to distinguish glycated from non-glycated proteins.

The term “monoclonal antibody,” as used herein, refers to an antibody from a substantially homogeneous population of antibodies that specifically binds to the same epitope. A monoclonal antibody may be secreted by a hybridoma or be produced using recombinant DNA technology (see, e.g., U.S. Pat. No. 4,816,567). A hybridoma can be produced according to certain methods known to those skilled in the art (see, e.g., Kohler et al., Nature (1975) 256: 495-499). A monoclonal antibody may also refer to an antibody fragment isolated from a phage display library (see, e.g., Clackson et al., Nature (1991) 352: 624-628; and Marks et al. J. Mol. Biol. (1991) 222:581-597). For various other monoclonal antibody production techniques, see, e.g., Harlow et al., Antibodies: A Laboratory Manual, Cold Spring Harbor Laboratory, Cold Spring Harbor, N.Y., 1988.

The term “epitope,” as used herein, refers to any polypeptide determinant capable of specifically binding to an antibody or a T-cell receptor. In certain embodiments, an epitope is a region of an antigen that is specifically bound by an antibody. An epitope may include chemically active surface groupings of molecules, such as amino acids, sugar side chains, phosphate, or sulfonyl groups. An epitope may also have specific three dimensional structural characteristics (e.g., a “conformational” epitope) and/or specific charge characteristics.

The term “polyclonal antibody,” as used herein, refers to a heterogeneous mixture of antibodies that recognize and bind to different epitopes on the same antigen. Polyclonal antibodies may be obtained from crude serum preparations or may be purified using, for example, antigen affinity chromatography, or Protein A/Protein G affinity chromatography.

A “sandwich assay” typically involves contacting a sample solution of an analyte (e.g., blood or urine) to a surface presenting a first binding material immunologically specific for that analyte. After an optional washing step, a solution comprising a labeled second binding material specifically reactive with the analyte is then added to the assay. The labeled second binding material will bind to any analyte which is itself bound to the first binding material. The assay system is then optionally subjected to a washing step to remove any labeled second binding material which failed to bind with the analyte. The amount of labeled second binding material remaining may then be determined and will be indicative of the amount of analyte present in the sample.

While the term “sandwich assay” is generally understood by those skilled in the art to relate to immunoassays wherein the first binding material and the labeled second binding material are both antibodies or are both antigens such that the “sandwich” is of the form of antibody/antigen/labeled antibody, a broader definition of the term “sandwich-type assay” is understood as including other types of three component assays including what are sometimes referred to as “indirect sandwiches,” which may be of the form of antigen/antibody/labeled (anti-immunoglobulin) antibody.

A “competitive binding assay” refers to an assay in which an analyte is detected and quantified by its ability to block the specific binding of a labeled, known material to its antibody. For example, a reagent that is known to bind to a target is provided. Usually, the reagent does not have desired pharmacological properties. The reagent is typically labeled with a labeling agent (such as fluorescein) to allow for detection. If the mixture of a compound and the reagent is incubated with the target, and if the compound is able to bind to the target, then the binding of the reagent to the target will be inhibited by the compound.

### BRIEF DESCRIPTION OF THE DRAWINGS

FIG. 1A shows an example of glycation prediction for ALDH1L1 (Table 1, entry 27) based on the NetGlycate-1.0 software (www.cbs.dtu.dk/services/NetGlycate-1.0; and Johansen et al., Glycobiol. (2006) 16:844) with the additional constraint requiring the protein to have at least one glycation potential score cutoff of >0.9 for at least one lysine residue. FIG. 1B shows the amino acid sequence of ALDH1L1. Only Lys²¹, Lys²⁸⁷, Lys³⁰⁷, and Lys⁶⁶⁹ have a glycation potential score of >0.9 (bold and underlined in FIG. 1B). His²⁴, bold and italicized in FIG. 1B, is analogous to the position of His⁴⁴ relative to glycated Lys⁴¹ in the glycation motif of CG59. (Acosta et al., Proc. Natl. Acad. Sci. USA (2000) 97:5450).

FIG. 2 shows the result of detection of glycated proteins in urine using a dot blot test. 50 μl of diluted urine (1:40) sample was spotted on a nitrocellulose membrane, air-dried, and then exposed to a primary antibody (rabbit anti-glucocytollysine mAb, 1 μg/ml), washed and then exposed to a secondary antibody (donkey anti-rabbit IgG tagged with the fluorescent probe IRdye800 (1:1000)). The intensity signal in each spot was quantified using a Li-Cor Odessy Infrared Scanner.

### DETAILED DESCRIPTION OF CERTAIN EMBODIMENTS OF THE INVENTION

The present invention provides for novel methods, compositions, and kits for detecting glycated proteins in a sample of bodily fluid (e.g., blood, urine, sweat, serum, and plasma) from a subject. The level of glycated proteins is elevated in diabetic patients. Thus, the methods, compositions, and kits of the present invention can be used to diagnose or follow a diabetic condition by detecting the levels of glycated proteins in a subject. The methods, compositions, and kits of the present invention can also be used to screen for a diabetic condition by detecting glycated proteins in a population of subjects.

Glycation involves the non-enzymatic reaction of reducing sugars (e.g., glucose) with amino groups in proteins, lipids, or other molecules. In contrast, glycosylation involves the enzymatic attachment of sugars to proteins, lipids, or other molecules. Examples of glycated proteins include those having a glycated α-amino group at the N-terminus (for example, glycated hemoglobin) and those in which the ε-amino group of lysine of a protein has been glycated (for example, glycated albumin). Those proteins, which bear one or more α-amino and/or ε-amino groups that can react preferentially and non-enzymatically with glucose, are appreciated by those skilled in the art as having a glycation motif.

Glycation of proteins is believed to represent the major mechanism by which high levels of glucose over time induce cellular and tissue damage in the target organs of diabetic subjects. Glycation of proteins depends on the glucose levels to which proteins are exposed. Because plasma glucose levels are a continuum, it is not surprising that glycated proteins are present in both non-diabetic and diabetic subjects, albeit at higher levels in diabetic subjects than in non-diabetic subjects. Thus, diagnosing and following a diabetic condition in a subject and screening a population of subjects for a diabetic condition can be achieved by detecting the level of glycated protein in a subject and/or a population of subjects.

The inventors previously appreciated that a specific glycated protein, i.e., glycated CD59, can be isolated from bodily fluids which typically comprise a mixture of different glycated proteins. The level of glycated CD59 can then be determined for diagnosing and following a diabetic condition. For details, see U.S. Pat. Nos. 6,835,545; 7,049,082; and 7,767,791; incorporated herein by reference.

Glycated proteins in urine can be detected to discriminate with a high degree of confidence non-diabetic from diabetic subjects. The inventive system does not require identifying or isolating the individual glycated proteins. Therefore, although the present invention may encompass isolating and/or purifying glycated proteins from urine, unprocessed urine can also be used in the present invention.

Glycated proteins are found in a variety of bodily fluids of a subject, such as in blood, urine, sweat, serum, saliva, and plasma. In certain embodiments, urine is the bodily fluid used as the sample from the subject. Glycated proteins present in urine include glycated CD59, glycated albumin, glycated hemoglobin, glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, and glycated apolipoprotein A4 (Ukita et al., Clin. Chem. (1991) 37:504; Johansen et al., Glycobiol. (2006) 16:844; and Davies et al., J. Exp. Med. (1989) 170:637). Any or a portion of the glycated proteins found in urine may be detected in the present invention. Therefore, one of the advantages of the present invention is that the detection of glycated proteins can be performed using a sample of urine without requiring a blood sample which requires a pin prick.

Because neither a blood sample nor a processed urine sample is required, the systems of present invention are easy-to-use and amenable to miniaturization for use in the setting of a doctor's office, in neighborhood clinics, and even at home. The possibility of using the present invention at points-of-care without drawing blood or needing a clinical laboratory to process a sample can help detect millions of undiagnosed diabetic subjects, especially in developing countries where access to medical services and clinical labs is not readily available. The present invention can assist with the early detection of diabetes and reduce the incidence and progression of diabetic complications. Combined, the benefits derived from the early detection of undiagnosed diabetes will reduce the staggering societal burden of diabetes.

In one aspect, provided is a method for diagnosing a diabetic condition in a subject comprising detecting one or more glycated proteins in a urine sample obtained from the subject. If the one or more glycated proteins are detected in the urine sample at a level above a threshold level, the subject is diagnosed with having a diabetic condition. If the one or more glycated protein(s) are not detected in the urine sample or are detected below a threshold level, the subject is identified as not having a diabetic condition. The threshold level may be a predetermined value. The threshold level may also be the level of glycated protein(s) in a control sample obtained from a subject or population of subjects. The control sample may be a sample obtained from a normal subject or population of normal subjects with a diabetic condition. In certain embodiments, the level of the one or more glycated protein(s) is obtained from a sample from the subject, and the control sample is a second sample from the subject obtained at a different time. In certain embodiments, the level of one or more glycated protein(s) in the control sample is a predetermined value, which can take a variety of forms. For example, the predetermined value can be a single cut-off value, such as a median or mean of a population of subjects. It can be established based upon comparative groups, such as one group having normal amounts of circulating insulin and another group having abnormal amounts of circulating insulin. Another example of comparative groups include one group with a condition and another group without the condition. The condition may be a diabetic condition. Another example of comparative groups include one group with a family history of a condition and another group without a family history of the condition. Again, the condition may be a diabetic condition. The predetermined value can be arranged, where a population is divided equally (or unequally) into groups, such as a low-risk group, a medium-risk group, and a high-risk group. The predetermined value will depend upon the particular population selected. For example, an apparently healthy population will have a different “normal” range than will a population which is known or thought to have a condition related to abnormal levels of glycated proteins, blood glucose, and/or insulin. Those different populations are herein called different categories. Accordingly, the predetermined value selected may take into account the category in which a subject falls. Appropriate ranges and categories can be selected with no more than routine experimentation by those of ordinary skill in the art. By abnormally high it is meant high relative to a selected control. Typically the control will be based on apparently healthy normal subjects which may be in a similar age bracket.

Proteins having a glycation motif may undergo glycation in different organs, tissues, and fluids of a subject to form glycated proteins. These glycated proteins or their glycated fragments may be released into the urine. Higher abundance of glycated proteins in urine may allow for easier detection. Therefore, although the present invention may involve detecting glycated proteins of relatively low abundance in urine, the present invention preferably involves detecting glycated proteins of higher abundance in urine. In certain embodiments, the one or more glycated proteins, such as glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, glycated apolipoprotein A4, or combinations thereof, are detected in the methods of the present invention. In addition to the above-identified glycated proteins, the one or more glycated proteins being detected may further include other glycated proteins present in urine. In certain embodiments, the glycated proteins further include glycated CD59. In some embodiments, the glycated proteins further include glycated albumin. In certain embodiments, the glycated proteins further include glycated immunoglobulins.

The glycated proteins being detected may be isolated and/or purified from a urine sample. Alternatively, the glycated proteins being detected need not be isolated or purified from the urine sample. In certain embodiments, the glycated proteins being detected include at least two glycated proteins. In certain embodiments, the glycated proteins being detected include at least three glycated proteins. In certain embodiments, the glycated proteins being detected include at least four glycated proteins. In certain embodiments, the glycated proteins being detected include at least five glycated proteins. In certain embodiments, the glycated proteins being detected include at least six glycated proteins. In certain embodiments, the glycated proteins being detected include at least seven glycated proteins. In certain embodiments, the glycated proteins being detected include at least eight glycated proteins. As understood by those skilled in the art, the glycated proteins being detected in urine may include even more glycated proteins.

A renal process involves the filtration of plasma in the glomerulus. Glomerular filtration involves substances moving from the lumen of the capillary, across the filtration membrane, and into Bowman's space. Low molecular weight substances pass more easily through the filtration membrane than high molecular weight substances. In certain embodiments, the glycated protein being detected in urine has a molecular weight less than approximately 50,000 g/mol. In certain embodiments, the glycated protein being detected has a molecular weight less than approximately 40,000 g/mol. In certain embodiments, the glycated protein being detected has a molecular weight less than approximately 30,000 g/mol. In certain embodiments, the glycated protein being detected has a molecular weight less than approximately 20,000 g/mol. In certain embodiments, the glycated protein being detected has a molecular weight less than approximately 10,000 g/mol. In certain embodiments, the glycated protein being detected has a molecular weight less than approximately 5,000 g/mol. The glycated protein being detected in a urine sample may also be of any other molecular weight as long as the glycated protein is able to penetrate the filtration membrane and is present in urine.

The foregoing glycated proteins may be degraded to give rise to various lower molecular weight fragments. At least some of those fragments may bear one or more glycated moieties formed through glycation reactions between one or more glycation motifs and glucose. The present invention may involve detecting fragments of the foregoing glycated proteins or other glycated proteins found in a subject.

The step of detecting may be performed using an antibody, or antigen-binding fragment thereof, that binds to one or more glycated proteins. The antibody, or antigen-binding fragment thereof, may bind to the one or more glycated proteins found in urine or other bodily fluids through non-covalent interactions such as hydrogen bonding, surface interpenetration, ionic bonding, van der Waals forces, hydrophobic interactions, dipole-dipole interactions, and combinations thereof.

The step of detecting may be performed using an antibody, or antigen-binding fragment thereof, that binds non-specifically to one or more glycated proteins or specifically to a glycated protein. In certain embodiments, the step of detecting is performed using an antibody that binds non-specifically to one or more glycated proteins. In certain embodiments, the step of detecting is performed using an antigen-binding fragment of an antibody wherein the antigen-binding fragment binds non-specifically to one or more glycated proteins.

In certain embodiments, the step of detecting is performed using an antibody that binds specifically to a glycated protein. In certain embodiments, the step of detecting is performed using an antigen-binding fragment of an antibody wherein the antigen-binding fragment binds specifically to a glycated protein. In certain embodiments, the antibody is an anti-glucitollysine antibody. In certain embodiments, the antibody is a rabbit anti-glucitollysine monoclonal antibody (e.g., Clone 42). Other antibodies that bind specifically to a glycated protein and may be useful in the invention include, but are not limited to, anti-glycated hemoglobin monoclonal antibodies; anti-glycated albumin antibodies; A717, a murine monoclonal antibody of the IgG1 class that selectively recognizes Amadori-modified albumin; mouse anti-glycated human hemoglobin HbA1c monoclonal antibodies; and anti-glycated LDL antibodies.

Amino groups in proteins are typically glycated under glycation conditions because, among other things, the amino groups are nucleophilic. Lysine and arginine residues in a protein have amino groups that may be glycated. Therefore, antibodies that bind to such glycated residues may be useful in the invention.

In certain embodiments, the step of detecting is performed using an antibody that binds to a glycated lysine residue of a glycated protein. In certain embodiments, the step of detecting is performed using an antigen-binding fragment of an antibody wherein the antigen-binding fragment binds to a glycated lysine residue of a glycated protein. In certain embodiments, the step of detecting is performed using an antibody that binds to a glycated arginine residue of a glycated protein. In certain embodiments, the step of detecting is performed using an antigen-binding fragment of an antibody wherein the antigen-binding fragment binds to a glycated arginine residue of a glycated protein.

In certain embodiments, the step of detecting is performed using an antibody that binds to the glycated N-terminus of a glycated protein. In certain embodiments, the step of detecting is performed using an antigen-binding fragment of an antibody wherein the antigen-binding fragment binds to the glycated N-terminus of a glycated protein.

As will be appreciated by those skilled in the art, the step of detecting may also be performed using an antibody, or antigen-binding fragment thereof, that binds to a glycated residue, other than a glycated lysine residue, glycated arginine residue, or glycated N-terminus of a glycated protein.

All forms, such as tautomers, stereoisomers, enantiomers, and diastereomers, of glycated protein(s) may be detected using the inventive methods. The present invention provides for detecting Amadori products of the glycated proteins. In certain embodiments, the Amadori products of the full-length glycated proteins may be detected. In certain embodiments, the present invention provides for detecting the Amadori products of fragments of the glycated proteins.

Glycated protein(s) have sugar rings or ketones on the glycated residues. Those sugar rings or ketones may be reduced to form open-chain polyalcohols. An antibody may then bind to the polyalcohol alone or additionally to the residue to which the polyalcohol is attached. For example, a glucose moiety attached to the amino group of a lysine residue of a glycated protein may be reduced into a glucitol moiety attached to the lysine residue. An anti-glucitollysine antibody may then be used to bind to the glucitollyl moiety, or an anti-glucitollysine antibody may be employed to bind to the glucitollysyl moiety. The reduction may be performed using an inorganic or organic reductant such as sodium borohydride.

The anti-glucitollysine antibody may be prepared in a mammal, such as a goat, rabbit, rat, or mouse, by immunization with glucitollysine coupled with various carrier proteins, as taught in Myint et al., Biochim. Biophys. Acta (1995) 1272:73-79, incorporated herein by reference. The anti-N^{E}-glucitollysine antibody and other antibodies binding to glycated proteins may be prepared in a similar way by those skilled in the art.

In certain embodiments, a fragment of anti-glucitollysine antibody may be used to bind to the glucitollysyl moiety in a glycated protein. In certain embodiments, a fragment of anti-glucitollysine antibody may be used to bind to the glucitollysyl moiety in a glycated protein.

The antibody useful for binding to glycated protein(s) in the present invention may be a monoclonal antibody, and the antigen-binding fragment may be a fragment of the monoclonal antibody. Alternatively, the antibody binding to the glycated protein(s) may be a polyclonal antibody.

In certain embodiments, the antibody, or antigen-binding fragment thereof, binds to a protein and not to the glycated proteins. Such antibody, or antigen-binding fragment thereof, may also be useful for detecting glycated proteins because of its ability to discriminate between glycated and non-glycated proteins. The antibody includes, but are not limited to, an antibody that binds specifically to a protein, an antibody that binds specifically to a fragment of a protein, an antibody that binds non-specifically to two or more proteins, an antibody that binds non-specifically to fragments of two or more proteins, an antibody that binds non-specifically to lysine-glycated regions of two or more proteins, an antibody that binds non-specifically to arginine-glycated regions of two or more proteins, and an antibody that binds non-specifically to N-terminal-glycated regions of two or more proteins. Certain antibodies known in the art and useful in the present invention include anti-CD59 antibody, for example, anti-CD-59 YTH53.1.

Significantly, as is well-known in the art, only a small portion of an antibody molecule, the paratope, is involved in the binding of the antibody to its epitope (see, e.g., Clark, The Experimental Foundations of Modem Immunology, Wiley & Sons, Inc., New York, 1986; Roitt, Essential Immunology, 7th Ed., Blackwell Scientific Publications, Oxford, 1991). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Furthermore, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (Frs), which maintain the tertiary structure of the paratope (see, e.g., Clark, The Experimental Foundations of Modem Immunology, Wiley & Sons, Inc., New York, 1986; Roitt, Essential Immunology, 7th Ed., Blackwell Scientific Publications, Oxford, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody (see, e.g., U.S. Pat. Nos. 4,816,567; 5,225,539; 5,585,089; 5,693,762; and 5,859,205).

Thus, for example, PCT International Publication No. WO 92/04381 teaches the production and use of humanized murine RSV antibodies in which at least a portion of the murine FR regions has been replaced by FR regions of human origin. Such antibodies, including fragments of intact antibodies with antigen-binding ability, are often referred to as “chimeric” antibodies.

In certain embodiments, the antibodies, or antigen-binding fragment thereof, useful in the present invention, include F(ab′)₂, Fab, Fv, and Fd fragments; chimeric antibodies in which the Fc, and/or Fr, and/or CDR1, and/or CDR2, and/or light chain CDR3 regions have been replaced by homologous human or non-human sequences; chimeric F(ab′)₂ fragment antibodies in which the FR, and/or CDR1, and/or CDR2, and/or light chain CDR3 regions have been replaced by homologous human or non-human sequences; chimeric Fab fragment antibodies in which the FR, and/or CDR1, and/or CDR2, and/or light chain CDR3 regions have been replaced by homologous human or non-human sequences; and chimeric Fd fragment antibodies in which the FR, and/or CDR1, and/or CDR2 regions have been replaced by homologous human or nonhuman sequences. The present invention also encompasses the use of single chain antibodies.

In certain embodiments, the antibody, or antigen-binding fragment thereof, includes polypeptides of various sizes and types that bind specifically to a glycated protein. These polypeptides may be useful to the present invention because of their ability to discriminate between glycated protein and non-glycated protein. These polypeptides may be derived from sources other than antibody technology. For example, such polypeptides can be provided by degenerate peptide libraries which can be readily prepared in solution, in immobilized form, or as phage display libraries. Combinatorial libraries also can be synthesized of peptides containing one or more amino acids. Libraries further can be synthesized of peptoids and non-peptide synthetic moieties.

Suitable assay methods for purposes of the present invention to detect glycated proteins in urine or other sample may comprise any of the assay formats known in the art. In certain embodiments, an immunoassay is used to detect glycated proteins in a sample. In certain embodiments, the immunoassay is a sandwich assay. In certain embodiments, the immunoassay is a sandwich-type assay. In certain embodiments, the immunoassay is a competitive binding assay. In certain embodiments, the immunoassay is a one-step direct test. In certain embodiments, the immunoassay is a two-step test.

In certain embodiments, a dot blot assay is used to detect glycated proteins in a sample. For example, the glycated proteins in urine or other sample need not first be isolated and/or purified. Instead, the sample containing the glycated proteins is applied directly to a membrane as a dot. The membrane may be a nitrocellulose, polyvinylidene difluoride, or nylon membrane. The glycated proteins are immobilized on the membrane through non-covalent or covalent interactions. The membrane is then contacted with at least one or more probes under suitable incubation and washing conditions. Finally the presence of probes bound to the membrane is detected. In certain embodiments, the amount of probes bound is quantifiable.

The one or more probes may include an antibody or antigen-binding fragment thereof. As described above, the antibody or antigen-binding fragment thereof may bind specifically to a glycated protein or non-specifically to one or more glycated proteins through non-covalent interactions.

After optionally washing away any material unbound to the membrane, the antibody, or antigen-binding fragment thereof, bound to the glycated proteins may be detected. To facilitate the detection of glycated proteins, the antibody, or antigen-binding fragment thereof, may be optionally coupled to one or more labeling agents for imaging of the glycated proteins to which the antibody, or antigen-binding fragment thereof, is bound. The antibody, or antigen-binding fragment thereof, may be coupled to, e.g., fluorescent labeling agents, radioactive labeling agents, enzymatic labeling agents, or biotin labeling agents. The labeling agents may be attached to the antibody, or antigen-binding fragment thereof, by reacting with an amino, carboxyl, thiol, or other nucleophilic or electrophilic group of the antibody, or antigen-binding fragment thereof. Fluorescent labeling agents useful in the invention include, e.g., amino-, carboxyl-, or thiol-reactive fluorescent dyes. Exemplary fluorescent dyes that may be useful in the invention are xanthene derivatives, cyanine derivatives, naphthalene derivatives, coumarin derivatives, oxadiazole derivatives, pyrene derivatives, oxazine derivatives, acridine derivatives, arylmethine derivatives, and tetrapyrrole derivatives. In certain embodiments, fluorescenyl, bromobimane, or Alexa fluor moieties are used as the fluorescent labeling agent. Biotin labeling agents useful in the invention include, e.g., amino-, carboxyl-, or thiol-reactive agents containing a biotin moiety. The antibody, or antigen-binding fragment thereof, may be coupled to one or more labeling agents prior or subsequent to binding to the protein of interest.

Means for detecting the labeling agents are well known to those of skill in the art. For example, where the labeling agent is a radioactive labeling agent, means for detection include a scintillation counter or photographic film as in autoradiography. Where the labeling agents are fluorescent labeling agents, they may be detected by exciting a fluorophore with an appropriate wavelength of light and detecting the resulting fluorescence. The fluorescence may be detected visually, by means of a photographic film, by the use of an electronic detector such as a charge coupled device (CCD) or photomultiplier, or by other means well known in the art. Similarly, enzymatic labeling agents may be detected by providing appropriate substrates for the enzymatic labeling agents and detecting the resulting reaction product.

In certain embodiments, glycated proteins may be detected using a reverse dot blot assay. For example, the glycated proteins contain, or are caused to contain, one or more labeling agents. In this format, probes that are not labeled with any labeling agent are bound to a solid support and exposed to the labeled glycated proteins under appropriate incubation and washing conditions. It is also to be understood that any other assay method, based on the formation of a complex or adduct between the glycated proteins in the sample and one or more probes, may be used.

The present invention involves a subject, or a population of subjects, having been diagnosed or not having been diagnosed with a diabetic condition. In certain embodiments, the subject is a mammal. In certain embodiments, the subject is a human. In certain embodiments, the subject has been previously diagnosed with a diabetic condition. In certain embodiments, the subject has not been previously diagnosed with a diabetic condition. The diabetic condition may be characterized by, e.g., abnormal levels of glycated proteins in the urine or blood, hyperglycemia, impaired glucose tolerance, insulin resistance, hepatic steatosis, non-alcoholic steatohepatitis (NASH), pediatric NASH, obesity, childhood obesity, metabolic syndrome, polycystic ovary disease, gestational diabetes, or combinations thereof. In certain embodiments, the diabetic condition is characterized by abnormal levels of glycated proteins in the urine or blood. In certain embodiments, the diabetic condition is characterized by hyperglycemia. In certain embodiments, the diabetic condition is characterized by hypoglycemia. In certain embodiments, the diabetic condition is characterized by impaired glucose tolerance. In certain embodiments, the diabetic condition is characterized by insulin resistance.

In certain embodiments, the subject has been previously diagnosed with a pre-diabetic condition. In certain embodiments, the subject has not been previously diagnosed with a pre-diabetic condition. Pre-diabetic conditions include, without limitation, abnormal levels of glycated proteins in the urine or blood, metabolic syndrome, impaired glucose tolerance, impaired fasting glycemia, and combinations thereof. In certain embodiments, the pre-diabetic condition is characterized by abnormal levels of glycated proteins in the urine.

In certain embodiments, the subject is at an increased risk of becoming diabetic. In certain embodiments, the subject is at an increased risk of becoming pre-diabetic (e.g., based on family history).

In certain embodiments, the subject is being treated to regulate blood sugar levels. In certain embodiments, the subject is being treated to regulate blood sugar levels. For example, the subject may be treated to regulate blood sugar levels using a non-drug or drug therapy. The drug therapy may be an oral blood sugar regulating agent therapy, an injectable drug therapy, insulin therapy, insulin analog therapy, or any other drug therapy known in the art.

In yet another aspect, the present invention provides for methods of diagnosing a diabetic condition in a subject. The method of diagnosing includes obtaining the level of one or more glycated proteins in a sample obtained from the subject and comparing the level of the one or more glycated proteins in the sample to a level from a control sample. If the level of the one or more glycated proteins in the sample exceeds the level of the one or more glycated proteins in the control sample, the subject is diagnosed with having a diabetic condition. In certain embodiments, the level is 1.1-fold, 1.2-fold, 1.5-fold, 2-fold, 3-fold, 5-fold, 10-fold, or more greater than a threshold level or a level found in a control sample. If the level of the one or more glycated proteins in the sample is below the level of the one or more glycated proteins in the control sample or the threshold level, the subject is not diagnosed with having a diabetic condition. The one or more glycated proteins being assayed for may be glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, glycated apolipoprotein A4, or combinations thereof.

In yet another aspect, the present invention provides for a method of following a diabetic condition in a subject. The method of following a subject's condition includes detecting one or more glycated proteins in a sample obtained from the subject. The detection of the one or more glycated proteins in the sample indicates onset, progression, or regression of a diabetic condition. The glycated proteins being assayed for may be glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, glycated apolipoprotein A4, or combinations thereof.

In certain embodiments, the step of detecting involves obtaining levels of glycated proteins in the sample from the subject and comparing levels of glycated proteins to a threshold level. In certain embodiments, the sample is a urine sample. For example, it is expected that an increase in the level of glycated proteins in the sample compared with the threshold level indicates a progression of a diabetic condition and/or poor glucose control, and that a decrease in the level of glycated proteins in the sample compared with the threshold level indicates a good glucose control and/or a regression of the diabetic condition. As understood by those of skill in the art, an abrupt increase in the level of glycated proteins in the sample compared with the threshold level indicates an onset of a diabetic condition.

Accordingly, one can monitor levels of glycated proteins in a sample of a subject over time to determine if the diabetic condition of the subject (e.g., blood glucose levels) is changing over time. For example, levels of glycated proteins in the sample of a subject over time can be monitored to determine if the glucose levels of the subject are properly controlled. Changes in relative or absolute levels of the glycated proteins in the sample of greater than 0.1% may be significant. The change in levels of glycated proteins in the sample may be greater than 0.2%, 0.5%, 1.0%, 2.0%, 3.0%, 4.0%, 5.0%, 7.0%, 10%, 15% 20%, 25%, 30%, 40%, 50%, or more.

In certain embodiments, level of glycated protein in the sample of a subject are measured in samples collected from the subject at different times, e.g., at 12 hours apart. The time to obtain the subsequent sample from the subject may be at least 1 day, 2 days, 4 days, 1 week, 2 weeks, 3 weeks, 1 month, 2 months, 3 months, 4 months, 5 months, or 6 months after the time of the previous sample collection.

The comparison of levels of glycated proteins in two or more samples, taken at different times, is a measure of level of the subject's glucose control and allows evaluation of the treatment to regulate blood sugar levels. The comparison of a subject's levels of glycated proteins measured in samples obtained at different times provides a measure of glucose control to determine the effectiveness of any treatment to regulate blood sugar levels.

In yet another aspect, the present invention provides for methods of screening a population of subjects for a diabetic condition. The method of screening includes detecting one or more glycated proteins in a sample obtained from each subject. If the glycated proteins are detected in a sample of a subject at a level above a threshold level, the subject is diagnosed with having a diabetic condition. If the glycated proteins are not detected in a sample of a subject at a level above a threshold level, the subject is not diagnosed with having a diabetic condition. In the present invention, the glycated proteins may be glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, glycated apolipoprotein A4, and combinations thereof.

In yet another aspect, the present invention provides for a composition containing an antibody, or antigen-binding fragment thereof, useful in detecting glycated proteins in a bodily fluid from a subject. In certain embodiments, the composition is useful in detecting glycated proteins in the urine or other bodily fluids from the subject.

In still another aspect, also provided is a diagnostic kit useful in performing the methods of the present invention. In certain embodiments, the kit is useful for detecting glycated proteins in urine, particularly unprocessed urine. In certain embodiments, the kit comprises one or more containers filled with one or more compositions, such as an antibody, or antigen-binding fragment thereof, which binds to glycated protein(s). In certain embodiments, the glycated protein(s) are found in the urine.

In certain embodiments, the kit comprises an antibody, or antigen-binding fragment thereof, that binds specifically to a glycated protein selected from the group consisting of glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, and glycated apolipoprotein A4.

In certain embodiments, the kit comprises an antibody, or antigen-binding fragment thereof, that binds to one or more glycated proteins selected from the group consisting of glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, and glycated apolipoprotein A4, wherein the antibody, or antigen-binding fragment thereof, does not bind to the non-glycated form of the proteins selected from the group consisting of immunoglobulins, hemopexin, vitamin D binding protein, fibrinogen alpha chain, apolipoprotein A1, transferrin, macroglobulin alpha 2, complement component 4A, fibrinogen beta chain, fibrinogen alpha chain, abhydrolase domain-containing protein 1 4B, amiloride-sensitive amine oxidase copper-containing precursor, angiotensin-converting enzyme isoform 1 precursor, peptidase family M2 Angiotensin converting enzyme, aconitase 1, lysosomal acid phosphatase isoform 1 precursor, pancreatitis-associated protein, alpha-actinin-4, metalloproteinase with thrombospondin type 1 motifs, aspartylglucosaminidase, adenosylhomocysteinase, alpha-2-HS-glycoprotein, alcohol dehydrogenase NADP⁺, aldo-keto reductase family 1, aldehyde dehydrogenase family 1 member L1, aldolase B fructose-bisphosphate, pancreatic amylase alpha 2A, and apolipoprotein A4. In the present invention, the glycated proteins being detected may also include glycated CD59, glycated albumin, glycated hemoglobin, or combinations thereof.

In certain embodiments, the kits of the invention include an approved therapeutic agent for treating or preventing a diabetic condition.

In certain embodiments, the kits includes instructions for using the antibody, or antigen-binding fragment thereof, to detect the glycated protein(s) in a sample (e.g., urine).

In certain embodiments, the kit includes instructions for using the antibody, or antigen-binding fragment thereof, to obtain the levels of the glycated proteins in a sample (e.g., urine).

### EXAMPLES

### Example 1

### Prediction of Glycated Proteins in Urine Using NetGlycate-1.0 Software

Glycated proteins such as human serum albumin, low-density liopoprotein, and CD59 (Table 1, entries 1-3, respectively) were reported in the literature (Ukita et al., Clin. Chem. (1991) 37:504; Johansen et al., Glycobiol. (2006) 16:844; and Davies et al., J. Exp. Med. (1989) 170:637). Those three literature references reported 1,823, 1,152, and 1,400 proteins in urine, respectively. By analyzing those three data sets, 658 proteins were identified to be common to all three studies (Marimuthu et al., J. Proteome. Res., (2011) 10:2734; Adachi et al., Genome. Biol. (2006) 7:R80; and Li et al., Rapid Commun. Mass Spectrom. (2010) 24:823). Analyzing a sample of these 658 proteins with NetGlycate-1.0 software (www.cbs.dtu.dk/services/NetGlycate-1.0; and Johansen et al., Glycobiol. (2006) 16:844) predicted proteins of enties 1, 9, and β-30 (Table 1) are likely to be glycated in urine. The analysis showed that any of the proteins of enties 1, 9, and β-30 have at least one glycation potential score cutoff of >0.9 for a least one lysine.

Out of the 658 proteins, proteins of entries 4-12 (Table 1) were already found to be glycated in plasma and erythrocytes (Marimuthu et al., J. Proteome. Res., (2011) 10:2734). It is thus likely that these glycated proteins are present in urine.

TABLE 1
List of exemplary glycated proteins present in urine

| Entry | Protein Description | Gene Symbol |
|---|---|---|
| 1 | Human serum albumin¹ | Alb¹ |
| 2 | Low-density liopoprotein¹ | LDL¹ |
| 3 | CD59⁴ | CD59 |
| 4 | Hemopexin² | HPX³ |
| 5 | Vitamin D binding protein² | GC³ |
| 6 | Fibrinogen, alpha chain² | FGA³ |
| 7 | Apolipoprotein A1² | APOA1³ |
| 8 | Transferin² | TF³ |
| 9 | Macroglobulin, alpha 2² | A2M³ |
| 10 | Complement component 4A² | C4A³ |
| 11 | Fibrinogen, beta chain² | FGB³ |
| 12 | Fibrinogen, alpha chain² | FGA³ |
| 13 | Abhydrolase domain-containing protein 1 4B | ABHD14B³ |
| 14 | Amiloride-sensitive amine oxidase [copper- | ABP1³ |
| | containing] precursor | |
| 15 | Angiotensin-converting enzyme isoform 1 | ACE³ |
| | precursor | |
| 16 | Peptidase family M2 Angiotensin converting | ACE2³ |
| | enzyme | |
| 17 | Aconitase 1 | ACO1³ |
| 18 | Lysosomal acid phosphatase isoform 1 precursor | ACP2³ |
| 19 | Pancreatitis-associated protein | ACPP³ |
| 20 | Alpha-actinin-4 | ACTN4³ |
| 21 | Metalloproteinase with thrombospondin type 1 | ADAMTS1³ |
| | motifs | |
| 22 | Aspartylglucosaminidase | AGA³ |
| 23 | Adenosylhomocysteinase | ACHY³ |
| 24 | Alpha-2-HS-glycoprotein | AHSG³ |
| 25 | Alcohol dehydrogenase [NADP⁺] | AKR1A1³ |
| 26 | Aldo-keto reductase family 1 | AKR1B1³ |
| 27 | Aldehyde dehydrogenase family 1 member L1 | ALDH1L1³ |
| 28 | Aldolase B, fructose-bisphosphate | ALDOB³ |
| 29 | Amylase, alpha 2A (pancreatic) | AMY2A³ |
| 30 | Apolipoprotein A4 | APOA4³ |

¹Ukita et al., Clin. Chem. (1991) 37: 504.
²Zhang et al., J. Proteome. Res., (2011) 10: 3076.
³Marimuthu et al., J. Proteome. Res., (2011) 10: 2734.
⁴Davies et al., J. Exp. Med. (1989) 170: 637.

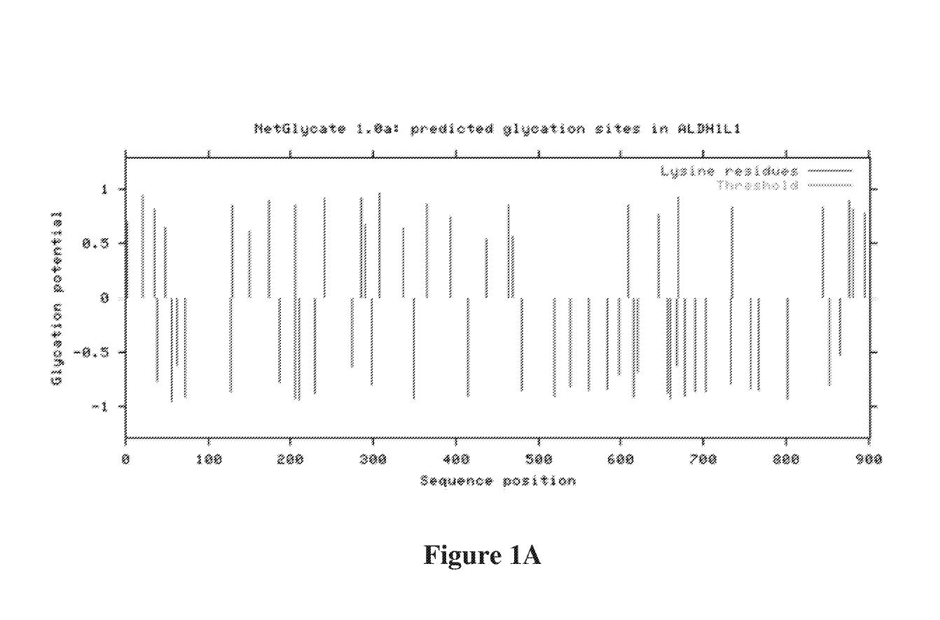

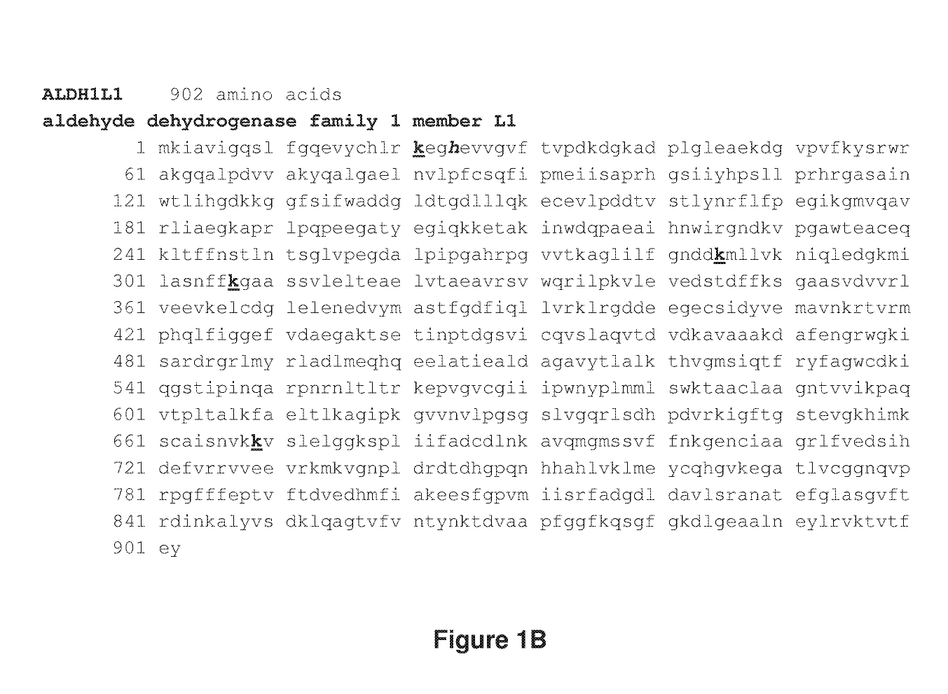

An example of the analysis is shown in FIG. 1 and Table 2, wherein the above-mentioned analysis was applied to aldehyde dehydrogenase family 1 member L1 (ALDH1L1, entry 27 in Table 1). The additional constraint imposed required the protein to have at least one glycation potential score cutoff of >0.9 for a least one lysine residue. Shown in FIG. 1A is the sequence of ALDH1L1. The result of the analysis showed that each of Lys²¹, Lys²⁸⁵, Lys³⁰⁷, and Lys⁶⁶⁹ of ALDH1L1 has a glycation potential score of >0.9 (Table 2; bold and underlined in FIG. 1B) and are considered very likely to be glycated in urine. His²⁴, bold and italicized in FIG. 1B, replicates the position of His⁴⁴ relative to glycated Lys⁴¹ in the glycation motif of CD59, which functions as a general acid/base catalyst during the Amadori rearrangement (Acosta et al., Proc. Nat. Acad. Soc., U.S.A. (2000) 97:5450).

TABLE 2
Prediction result of ALDH1L1 using netglycate-1.0 software

| Sequence # of Lys | Score |
|---|---|
| 2 | 0.704 |
| 21 | 0.945 |
| 35 | 0.821 |
| 38 | −0.772 |
| 48 | 0.644 |
| 55 | −0.960 |
| 62 | −0.624 |
| 72 | −0.924 |
| 128 | −0.870 |
| 129 | 0.855 |
| 150 | 0.610 |
| 174 | 0.900 |
| 187 | −0.788 |
| 205 | −0.928 |
| 206 | 0.862 |
| 210 | −0.948 |
| 229 | −0.889 |
| 241 | 0.915 |
| 274 | −0.643 |
| 285 | 0.920 |
| 290 | 0.678 |
| 298 | −0.811 |
| 307 | 0.971 |
| 337 | 0.645 |
| 349 | −0.926 |
| 365 | 0.870 |
| 394 | 0.750 |
| 415 | −0.904 |
| 437 | 0.541 |
| 463 | 0.861 |
| 469 | 0.569 |
| 479 | −0.856 |
| 520 | −0.906 |
| 539 | −0.817 |
| 561 | −0.864 |
| 583 | −0.852 |
| 597 | −0.713 |
| 608 | 0.853 |
| 615 | −0.920 |
| 620 | −0.690 |
| 645 | 0.767 |
| 656 | −0.885 |
| 660 | −0.931 |
| 668 | −0.629 |
| 669 | 0.937 |
| 677 | −0.912 |
| 690 | −0.875 |
| 703 | −0.873 |
| 733 | −0.792 |
| 735 | 0.829 |
| 757 | −0.845 |
| 767 | −0.857 |
| 802 | −0.934 |
| 845 | 0.836 |
| 852 | −0.805 |
| 865 | −0.531 |
| 876 | 0.899 |
| 882 | 0.818 |
| 896 | 0.780 |

### Example 2

### Dot Blot Test of Glycated Proteins in Urine Samples of Human Subjects

Three groups of fasting human subjects were tested. The first group consisted of five normal individuals. The second group consisted of five individuals showing impaired glucose tolerance (IGT). The third group consisted of five diabetic individuals.

Blood samples were drawn from each subject and were tested to obtain the level of glycated hemoglobin (HbA1c), the glucose level in a fasting plasma glucose (FG) test, the glucose level in an oral glucose tolerance test (OGTT). A urine sample was also obtained from the same subject and was assayed in a dot blot test.

### Glycated Hemoglobin Test on Blood Samples of Human Subjects

Assays that quantify HbA1c in blood samples are extensively used to monitor glycemic load and response to treatment in diabetic patients. In a glycated hemoglobin test, a blood sample of a human subject was drawn, and the percentage of HbA1c in the blood sample (hereinafter “HbA1c level”) was determined. An HbA1c level less than 6.5% is considered normal. An HbA1c level no less than 6.5% indicates a diabetic or an IGT condition.

### Fasting Plasma Glucose Test on Blood Samples of Human Subjects

In an FG test, a human subject fasted for at least 8 hours. A blood sample of the human subject was then drawn, and the fasting plasma glucose level (hereinafter “FG level”) was determined. Typically, the FG level of a normal human subject is less than about 110 milligrams per deciliter (mg/dl). An FG level between about 110 mg/dl and about 126 mg/dl indicates an IGT condition, and an FG level of more than about 126 mg/dl indicates a diabetic condition.

### Oral Glucose Tolerance Test on Blood Samples of Human Subjects

In an OGTT, a dose of 75 g glucose was administered orally to a human subject. After 2 hours post administering of the dose, the level of plasma glucose of the human subject (hereinafter “OGTT level”) was determined. An OGTT level below 140 mg/dl is typically observed for a normal human subject. An OGTT level between 140 mg/dl and 200 mg/dl indicates an IGT condition. An OGTT level above 200 mg/dl usually confirms a diagnosis of diabetes.

### Dot Blot Test on Urine Samples of Human Subjects

In a dot blot test, 2 μL of 1:40 dilution of a urine sample from a human subject in water was spotted on a nitrocellulose membrane. The spot containing the urine sample was treated with sodium borohydride to reduce the post-translationally glucose-modified lysine residues of the proteins into glucitollysyl residues that are recognized by the primary anti-glucitollysine rabbit mAb (clone 42). The membrane was then blotted with the primary antibody anti-glucitollysine rabbit mAb (clone 42) (1 μg/mL).

The membrane was next incubated with a secondary antibody donkey anti-rabbit IgG tagged with the fluorescent probe IRdye800 (1:1000).

A Li-Cor Odessy Infrared Scanner was used to detect the adduct of the primary antibody and the secondary antibody.

The primary anti-glucitollysine rabbit mAb (clone 42) and the secondary antibody donkey anti-rabbit IgG IRdye800 were prepared according to methods well known in the art.

### Results

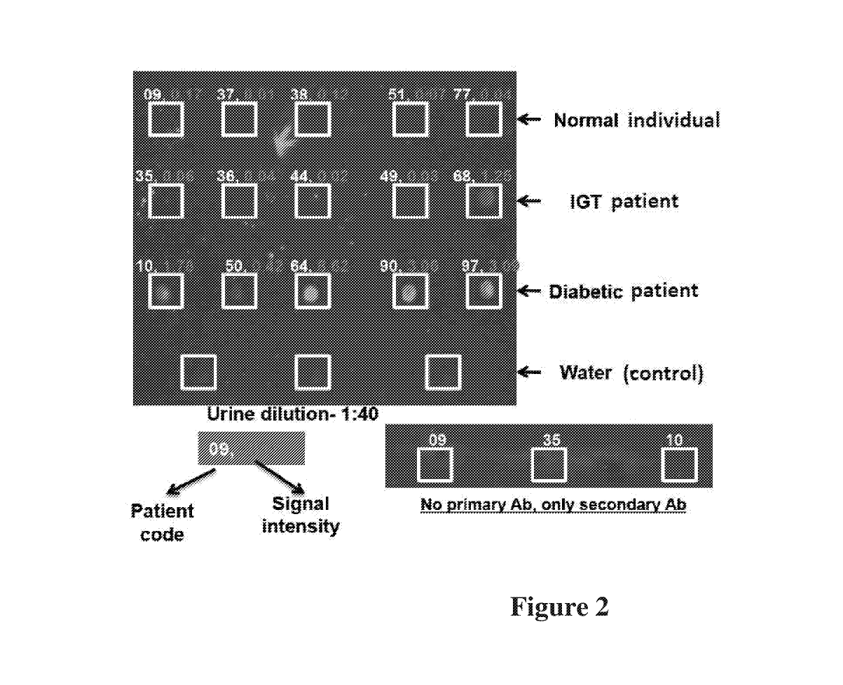

Summarized in Tables 3-5 are the results of the four different assays on urine and blood samples of normal human subjects, human subjects showing an IGT condition, and diabetic human subjects, respectively. The result of the dot blot test is also depicted in FIG. 2.

Of the samples of the normal human subjects tested (Table 3), all five blood samples showed HbA1c levels of <6.5%, fasting glucose (FG) levels of <110 mg/dl, and oral glucose tolerance test (OGTT) levels of <140 mg/dl, which are consistent with a normal, non-impaired glucose test (non-IGT), and non-diabetic condition.

In the dot blot test, of the five urine samples of the normal human subjects, four urine samples showed dot blot levels of about 0 fluorescence intensity unit, and only one urine sample showed a dot blot level of 0.17 fluorescence intensity unit. These dot blot levels were so low as to indicate low concentrations of the adduct of the primary antibody and the secondary antibody on the membrane and in turn low concentrations of glycated proteins in the urine samples, suggesting a normal, non-IGT, and non-diabetic condition.

TABLE 3
Result of blood and urine tests on normal human subjects

| | | | | Dot blot level |
|---|---|---|---|---|
| | HbA1c level | FG level | OGTT level | (fluorescence |
| Human subject | (%) | (mg/dl) | (mg/dl) | intensity unit) |
| 0255AGHA009 | 5.2 | 86 | 76 | 0.17 |
| 0255AGWS037 | 4.7 | 98 | 101 | −0.01 |
| 0255AGBJ038 | 4.9 | 91 | 72 | −0.12 |
| 0255AGDC051 | 4.6 | 81 | 103 | −0.07 |
| 0255AGSE077 | 5 | 96 | 92 | 0.04 |
| Mean | 4.88 | 90.4 | 88.8 | 0 |
| SD | 0.24 | 7 | 14.2 | 0.11 |
| CV | 4.90% | 7.80% | 16.00% | NA |
| Mean − 2SD | 4.4 | 76 | 60 | −0.22 |
| Mean + 2SD | 5.36 | 104 | 117 | 0.23 |

Of the blood samples of the human subjects having an IGT condition (Table 4), all five blood samples showed HbA1c levels of <6.5% and FG levels of ≦110 mg/dl. Those HbA1c and FG levels were also not significantly higher than the corresponding HbA1c and FG levels of blood samples obtained from normal human subjects, indicating that the HbA1c test or the FG test may not be capable of detecting an IGT condition. In contrast, all five blood samples showed OGTT levels of >140 mg/dl, suggesting that OGTT may be used to detect an IGT condition.

In the dot blot test, of the five urine samples of the human subjects having IGT, four urine samples showed dot blot levels ≦0.06 fluorescence intensity unit. However, one urine sample showed a dot blot level of 1.25 fluorescence intensity units, a dot blot level significantly higher than the dot blot levels of urine samples obtained from the normal human subjects, indicating a high concentration of the adduct of the primary antibody and the secondary antibody on the membrane and in turn a high concentration of glycated proteins in that urine sample. This result suggests that the dot blot test of the present invention may be used to detect an IGT condition.

TABLE 4
Result of blood and urine tests on human subjects
showing impaired glucose tolerance

| | | | | Dot blot level |
|---|---|---|---|---|
| | HbA1c level | FG level | OGTT level | (fluorescence |
| Human subject | (%) | (mg/dl) | (mg/dl) | intensity unit) |
| 0255AGWC035 | 5.4 | 87 | 176 | 0.06 |
| 0255AGRP036 | 5.1 | 97 | 194 | 0.04 |
| 0255AGDT044 | 5.8 | 110 | 180 | 0.02 |
| 0255AGDD049 | 5.4 | 90 | 191 | 0.03 |
| 0255AGZR068 | 5.5 | 76 | 181 | 1.25 |
| Mean | 5.44 | 92 | 184.4 | 0.28 |
| SD | 0.25 | 12.6 | 7.7 | 0.54 |
| CV | 4.60% | 13.70% | 4.20% | 194% |
| Mean − 2SD | 4.94 | 67 | 169 | −0.8 |
| Mean + 2SD | 5.94 | 117 | 200 | 1.36 |

Of the five blood samples of the diabetic human subjects (Table 5), three blood samples showed HbA1c levels of ≧6.5%, but two blood samples showed HbA1c levels of <6.5%. In the FG test, two blood samples showed FG levels of ≧126 mg/dl, but three blood samples showed FG levels of ≦117 mg/dl. These results indicate that the HbA1c test or the FG test may not be capable of detecting a diabetic condition. On the contrary, all five blood samples showed OGTT levels of >200 mg/dl, suggesting that OGTT may be used to detect a diabetic condition.

In the dot blot test, of the five urine samples of the diabetic human subjects, four urine samples showed dot blot levels of ≧1.78 fluorescence intensity units, and one urine sample showed a dot blot level of 0.42 fluorescence intensity unit. Those dot blot levels are significantly higher than the dot blot levels of urine samples obtained from the normal human subjects or human subjects having IGT, indicating high concentrations of the adduct of the primary antibody and the secondary antibody on the membrane and in turn high concentrations of glycated proteins in the urine samples. This result suggests that the dot blot test of the present invention is able to be used to detect a diabetic condition.

TABLE 5
Result of blood and urine tests on diabetic human subjects

| | | | | Dot blot level |
|---|---|---|---|---|
| | HbA1c level | FG level | OGTT level | (fluorescence |
| Human subject | (%) | (mg/dl) | (mg/dl) | intensity unit) |
| 0255AGYP010 | 5.3 | 99 | 244 | 1.78 |
| 0255AGTM050 | 6.5 | 117 | 208 | 0.42 |
| 0255AGSG064 | 5.7 | 113 | 230 | 8.62 |
| 0255AGCB090 | 6.8 | 137 | 292 | 3.6 |
| 0255AGRG097 | 7.8 | 126 | 233 | 3.09 |
| Mean | 6.42 | 118.4 | 241.4 | 3.5 |
| SD | 0.98 | 14.2 | 31.2 | 3.12 |
| CV | 15.20% | 12.00% | 12.90% | 89.00% |
| Mean − 2SD | 4.46 | 90 | 179 | −2.73 |
| Mean + 2SD | 8.38 | 147 | 304 | 9.73 |

The foregoing written specification is considered to be sufficient to enable one skilled in the art to practice the present invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the present invention are not necessarily encompassed by each embodiment of the invention.

All publications and patents mentioned herein are hereby incorporated by reference in their entirety for disclosure of the teachings relevant to the present invention, as if each individual publication or patent was specifically and individually indicated to be incorporated by reference. In case of the present specification and a document incorporated by reference including conflicting disclosure, the present specification shall control.

What is claimed is:

1. A method for diagnosing a diabetic condition in a subject comprising:
detecting one or more glycated proteins in a urine sample obtained from the subject, wherein one or more glycated proteins are selected from the group consisting of glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, and glycated apolipoprotein A4, and
wherein if one or more glycated proteins are detected in the urine sample, the subject is diagnosed with having a diabetic condition, or if one or more glycated proteins are not detected in the urine sample, the subject is not diagnosed with having a diabetic condition.

2. The method of claim 1, wherein one or more glycated proteins comprise two or more glycated proteins.

3. The method of claim 1, wherein one or more glycated proteins comprise three or more glycated proteins.

4. The method of claim 1, wherein one or more glycated proteins comprise four or more glycated proteins.

5. The method of claim 1, wherein one or more glycated proteins comprise five or more glycated proteins.

6. The method of claim 1, wherein one or more glycated proteins further comprise glycated CD59.

7. The method of claim 1, wherein one or more glycated proteins further comprise glycated albumin.

8. The method of claim 1, wherein one or more glycated proteins further comprise glycated hemoglobin.

9. The method of claim 1, wherein the glycated protein has a molecular weight less than approximately 30,000 g/mol.

10. The method of claim 1, wherein the glycated protein has a molecular weight less than approximately 20,000 g/mol.

11. The method of claim 1, wherein the step of detecting is performed using an antibody, or antigen-binding fragment thereof, that binds non-specifically to one or more glycated proteins.

12. The method of claim 11, wherein the antibody is anti-glucitollysine antibody.

13. The method of claim 11, wherein the antibody is a rabbit anti-glucitollysine monoclonal antibody (Clone 42).

14. The method of claim 1, wherein the step of detecting is performed using an antibody or antigen-binding fragment thereof, that binds specifically to a glycated protein.

15. The method of claim 13, wherein the antibody is selected from the group consisting of anti-glycated hemoglobin monoclonal antibodies, anti-glycated albumin antibodies, A717, mouse anti-glycated human hemoglobin HbA1c monoclonal antibody, and anti-glycated LDL antibodies.

16. The method of claim 1, wherein the step of detecting is performed using an antibody, or antigen-binding fragment thereof, that binds to a glycated lysine residue of the glycated protein.

17. The method of claim 16, wherein the antibody is anti-glucitollysine antibody.

18. The method of claim 16, wherein the antibody is rabbit anti-glucitollysine monoclonal antibody (Clone 42).

19. The method of claim 1, wherein the antibody is a monoclonal antibody.

20. The method of claim 1, wherein the antigen-binding fragment is a fragment of a monoclonal antibody.

21. The method of claim 1, wherein the antibody is a polyclonal antibody.

22. The method of claim 1, wherein the antibody is a polyclonal anti-glucitollysine antibody.

23. The method of claim 1, wherein the step of detecting is performed using an immunoassay.

24. The method of claim 23, wherein the immunoassay is selected from the group consisting of sandwich-type assays, competitive binding assays, one-step direct tests, and two-step tests.

25. The method of claim 23, wherein the step of detecting is performed using a dot blot assay.

26. The method of claim 1, wherein the subject has not been previously diagnosed with a diabetic condition.

27. The method of claim 1, wherein the subject has been previously diagnosed with a diabetic condition.

28. The method of claim 25, wherein the subject is being treated to regulate blood sugar levels.

29. The method of claim 1, wherein the diabetic condition is characterized by abnormal levels of glycated proteins in the urine.

30. A method for diagnosing a diabetic condition in a subject comprising:
obtaining the level of one or more glycated proteins in a urine sample obtained from the subject, wherein one or more glycated proteins comprise at least one glycated protein selected from the group consisting of glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, and glycated apolipoprotein A4, and
wherein if the level of one or more glycated proteins exceeds a threshold level, the subject is diagnosed with having a diabetic condition, or if the level of one or more glycated proteins is below the threshold level, the subject is not diagnosed with having a diabetic condition.

31. A method for diagnosing a diabetic condition in a subject comprising:
obtaining the level of one or more glycated proteins in a urine sample obtained from the subject, wherein one or more glycated proteins are selected from the group consisting of glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, and glycated apolipoprotein A4; and
comparing the level of one or more glycated proteins in the urine sample to a level from a control sample, wherein if the level of one or more glycated proteins in the urine sample exceeds the level of one or more glycated proteins in the control sample, the subject is diagnosed with having a diabetic condition, or if the level of one or more glycated proteins in the urine sample is below the level of one or more glycated proteins in the control sample, the subject is not diagnosed with having a diabetic condition.

32. A method for following a diabetic condition in a subject comprising:
obtaining the level of one or more glycated proteins in a urine sample obtained from the subject, wherein one or more glycated proteins are selected from the group consisting of glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, and glycated apolipoprotein A4; and
comparing the level of the one or more glycated proteins to a previous level as a determination of regression, progression or onset of the condition, wherein a higher level of the one or more glycated proteins in the urine sample compared to a previous level indicates the onset or progression of the condition, and wherein a lower level of the one or more glycated proteins in the urine sample compared to a previous level indicates regression of the condition.

33. A method for screening a population of subjects for a diabetic condition comprising:
detecting one or more glycated proteins in a urine sample obtained from each subject, wherein one or more glycated proteins are selected from the group consisting of glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, and glycated apolipoprotein A4, and
wherein if the glycated proteins are detected in a sample of a subject at a level above a threshold level, the subject is diagnosed with having a diabetic condition, and if the glycated proteins are not detected in a sample of a subject at a level above a threshold level, the subject is not diagnosed with having a diabetic condition.

34. A kit useful in performing the method of any one of claims 1-31, the kit comprising an antibody, or antigen-binding fragment thereof, that binds specifically to a glycated protein selected from the group consisting of glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, and glycated apolipoprotein A4.

35. A kit useful in performing the method of any one of claims 1-31, the kit comprising an antibody, or antigen-binding fragment thereof, that binds to one or more glycated proteins selected from the group consisting of glycated immunoglobulins, glycated hemopexin, glycated vitamin D binding protein, glycated fibrinogen alpha chain, glycated apolipoprotein A1, glycated transferrin, glycated macroglobulin alpha 2, glycated complement component 4A, glycated fibrinogen beta chain, glycated fibrinogen alpha chain, glycated abhydrolase domain-containing protein 1 4B, glycated amiloride-sensitive amine oxidase copper-containing precursor, glycated angiotensin-converting enzyme isoform 1 precursor, glycated peptidase family M2 Angiotensin converting enzyme, glycated aconitase 1, glycated lysosomal acid phosphatase isoform 1 precursor, glycated pancreatitis-associated protein, glycated alpha-actinin-4, glycated metalloproteinase with thrombospondin type 1 motifs, glycated aspartylglucosaminidase, glycated adenosylhomocysteinase, glycated alpha-2-HS-glycoprotein, glycated alcohol dehydrogenase NADP⁺, glycated aldo-keto reductase family 1, glycated aldehyde dehydrogenase family 1 member L1, glycated aldolase B fructose-bisphosphate, glycated pancreatic amylase alpha 2A, and glycated apolipoprotein A4,
wherein the antibody, or antigen-binding fragment thereof, does not bind to any of non-glycated proteins selected from the group consisting of non-glycated immunoglobulins, non-glycated hemopexin, non-glycated vitamin D binding protein, non-glycated fibrinogen alpha chain, non-glycated apolipoprotein A1, non-glycated transferrin, non-glycated macroglobulin alpha 2, non-glycated complement component 4A, non-glycated fibrinogen beta chain, non-glycated fibrinogen alpha chain, non-glycated abhydrolase domain-containing protein 1 4B, non-glycated amiloride-sensitive amine oxidase copper-containing precursor, non-glycated angiotensin-converting enzyme isoform 1 precursor, non-glycated peptidase family M2 Angiotensin converting enzyme, non-glycated aconitase 1, non-glycated lysosomal acid phosphatase isoform 1 precursor, non-glycated pancreatitis-associated protein, non-glycated alpha-actinin-4, non-glycated metalloproteinase with thrombospondin type 1 motifs, non-glycated aspartylglucosaminidase, non-glycated adenosylhomocysteinase, non-glycated alpha-2-HS-glycoprotein, non-glycated alcohol dehydrogenase NADP⁺, non-glycated aldo-keto reductase family 1, non-glycated aldehyde dehydrogenase family 1 member L1, non-glycated aldolase B fructose-bisphosphate, non-glycated pancreatic amylase alpha 2A, and non-glycated apolipoprotein A4.

36. The kit of claim 34 or 35, further comprising instructions for using the antibody, or antigen-binding fragment thereof, to detect the glycated proteins.

37. The kit of claim 34 or 35, further comprising instructions for using the antibody, or antigen-binding fragment thereof, to obtain the levels of the glycated proteins.
